(19) United States
(12) Patent Application Publication
(10) Pub. No.: US 2025/0236437 A1
(43) Pub. Date: Jul. 24, 2025

(54) STACKED MIRROR TRAY

(71) Applicant: EDWARDS LIFESCIENCES CORPORATION, Irvine, CA (US)

(72) Inventors: Tito Manuel Cairo Nunez, Tustin, CA (US); Deidania Karina Paredes Feliz, Lake Forest, CA (US); Said Pashtoun Sadat, Temecula, CA (US)

(73) Assignee: EDWARDS LIFESCIENCES CORPORATION, Irvine, CA (US)

(21) Appl. No.: 19/036,626

(22) Filed: Jan. 24, 2025

Related U.S. Application Data
(63) Continuation of application No. PCT/US2023/071233, filed on Jul. 28, 2023.
(60) Provisional application No. 63/371,196, filed on Aug. 11, 2022.

(51) Int. Cl. B65D 21/02

(52) U.S. Cl. CPC B65D 21/023; B65D 2585/54

(57) ABSTRACT

A packaging system includes a stackable tray for storing one or more implements. The stackable tray has one or more first interlocking features at a first surface of the tray and one or more second interlocking features at a second surface of the tray. A first stackable tray is arranged to mate with a second stackable tray via engagement of the first interlocking features or the second interlocking features when the first stackable tray is rotated from a first orientation to a second orientation.

### RELATED APPLICATION

This application is a continuation of International Patent Application No. PCT/US23/71233, filed Jul. 28, 2023, which claims the benefit of U.S. Provisional Patent Application Ser. No. 63/371,196, filed on Aug. 11, 2022, the complete disclosures of which are hereby incorporated by reference in their entireties.

### BACKGROUND

### Field

The present disclosure generally relates to the field of packaging.

### Description of Related Art

Often, products such as sterile surgical instruments are packaged within aseptic containers to be transported and/or stored in non-sterile environments. When a sterile instrument is to be used, the instrument (often within a package) is passed through a sterile barrier and into a sterile surgical field. Passing an instrument through the sterile barrier presents an opportunity for contamination of the surgical field. Further, storage space for sterile instruments within the surgical field is generally limited.

### SUMMARY

Described herein are methods, systems, and devices that relate to packaging, and specifically to sterile packaging for surgical instruments intended to be used in a sterile surgical field. In particular, various packaging solutions including stackable trays are disclosed that provide packaging for one or more sterile instruments in a single stackable tray as well as for additional instruments in a stack of like stackable trays. In some examples, a stackable tray includes interlocking features that can be employed to retain a quantity of stackable trays in a stack having a limited footprint. For example, interlocking features of a first stackable tray can engage with interlocking features of a second stackable tray to retain the first and second stackable trays in a stack. In some examples, the interlocking features of the first stackable tray engage with interlocking features of the second stackable tray when one of the stackable trays is rotated a predetermined rotational angle relative to the other stackable tray. Passing a stack of stackable trays through the sterile barrier increases the quantity of instruments passed through the sterile barrier in a single pass, thus reducing the opportunity for contamination of the surgical field. The stack of stackable trays can have a footprint equal to the footprint of a single tray to minimize storage space within the surgical field.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 is a perspective top view of an example stackable tray, according to one or more examples.

FIG. 2 is a perspective bottom view of an example stackable tray, according to one or more examples.

FIG. 3 is a side view of an example stackable tray, according to one or more examples.

FIG. 4 is an end view of an example stackable tray, according to one or more examples.

FIG. 5 is another end view of an example stackable tray, according to one or more examples.

FIG. 6 is a profile view of an example male interlocking feature and an example female interlocking feature, according to one or more examples.

FIG. 7 is a perspective top view of an example stack of stackable trays, according to one or more examples.

FIG. 8 is a perspective bottom view of an example stack of stackable trays, according to one or more examples.

FIG. 9 is a perspective top view illustrating the stacking of two stackable trays, according to one or more examples.

FIG. 10 is a side view of an example stack of stackable trays, according to one or more examples.

FIGS. 11 and 12 illustrate removing a stackable tray from stack of stackable trays, according to one or more examples.

FIG. 13 is a perspective top view of an example cover for a stackable tray, according to one or more examples.

FIG. 14 is a perspective bottom view of an example cover for a stackable tray, according to one or more examples.

FIG. 15 is a perspective top view of an example cover on a stackable tray. according to one or more examples.

FIG. 16 is a perspective top view of an example cover being aligned for placement on a stackable tray, according to one or more examples.

FIG. 17 is a perspective view of an example strap for a stack of stackable trays, according to one or more examples.

FIG. 18 is a side view of an example strap for a stack of stackable trays, according to one or more examples.

FIG. 19 illustrates an example flow chart for a process of stacking stackable trays, according to one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, various examples may be carried out in a manner that achieves or optimizes one advantage or group of advantages as taught herein without necessarily achieving other aspects or advantages as may also be taught or suggested herein.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” “distal,” “proximal,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that are similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a ,’ 10′ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

The present disclosure relates to systems, devices, and methods for packaging tools, instruments, implements, and the like, and/or associated components configured for use within a sterile surgical field. In some cases, the systems, devices, and methods may also be used for packaging non-sterile items. Sterile devices and non-sterile items can be advantageously packaged for short or long-term storage within a non-sterile environment. The terms “associated” and “associated with” are used herein according to their broad and ordinary meanings. For example, where a first feature, element, component, device, or member is described as being “associated with” a second feature, element, component, device, or member, such description should be understood as indicating that the first feature, element, component, device, or member is or is configured to be physically coupled, attached, or connected to, integrated with, used with, embedded at least partially within, or otherwise physically related to the second feature, element, component, device, or member, whether directly or indirectly.

As described below, sterile surgical instruments can be used to assist in the treatment or care of a patient within a sterile surgical field. Products such as sterile surgical instruments are often packaged within aseptic containers to be transported and/or stored in non-sterile environments. For instance, the aseptic containers with the instruments may be further packaged into boxes, pouches, and/or other containers to be shipped to various locations or to be warehoused until used.

Several layers of packaging may be used to retain the sterile nature of the instruments and to minimize or eliminate the possibility of contamination as the instruments are eventually introduced into the sterile surgical field. In many cases, surgical instruments may be packaged individually in sterile packages, then multiple individually packaged units may be packaged in larger containers, which also may be further packaged in outer containers, and so forth.

When the surgical instruments are to be used within the sterile surgical field, they are passed through a sterile “barrier” that separates the sterile surgical field from its surrounding environment. For instance, after removing some outer packaging, inner packaging can be opened in a controlled germ-free environment and then the sterile individual packaging can be passed through the sterile barrier and into the surgical field. Once in the surgical field, individual sterile packaging can be opened to access the instruments.

Each time an item is passed through the sterile barrier and into the surgical field, the opportunity exists for contamination of the surgical field. Accordingly, the greater the number of times that items are passed through the barrier, the greater the chances that some contamination, or a greater amount of contamination is introduced into the surgical field. Thus, it is desirable to reduce the number of times that the sterile barrier is breached. Further, it can be desirable to pass as many items as possible through the sterile barrier during each opening of the barrier to further minimize contamination. However, the area within the sterile surgical field is limited and space is often valuable.

Due to the limited accessibility and rigorous environmental conditions typically associated with a sterile surgical field, only certain types of packaging may be suitable for introduction into the surgical field. The systems, devices, and methods disclosed herein are suitable and advantageously employed within the sterile surgical field. As disclosed with various examples, the systems, devices, and methods disclosed herein can be used to package one or more sterile instruments in a single sterile package as well as to package multiple instruments in a stack of two or more sterile packages in a manner so as to manage aseptic presentation of the instruments within the sterile surgical field. Additionally, the use of the disclosed systems, devices, and methods can reduce contamination due to multiple transfers of products through the sterile barrier, while also limiting the footprint of the package or stack of packages within the surgical field.

The disclosed concepts may be adapted to any packaging application where the desire for sterile barrier management and a minimal footprint are regarded. Additionally, the disclosed concepts may also be applied to any packaging application where a single component or multiple components are to be packaged in a single package and where a stack of two or more such packages having a minimal footprint is desired.

A novel tray design is disclosed to allow multiple trays to be stacked and interlocked together, while maintaining the minimal footprint of a single tray. Each subsequent tray in a stack includes multiple interlock features at or near the edges of the tray. The interlock features are either female interlock features that comprise a recess in the topside surface of the tray or male interlock features that protrude below the lower surface of the tray. The interlock features on each tray are either disposed on a raised portion of the tray edge or a recessed portion of the tray edge, relative to each other, in order to reach subsequently stacked trays, either above or below a given tray. In some cases, “raised portions” of the tray edge can be level with the overall surface of the tray, with the lower portions of the tray edge being recessed relative to the overall surface of the tray, as shown in the accompanying illustrations. Trays can be made in various perimeter shapes. In many cases, regardless of the perimeter shape of the tray, each tray can be identical.

To stack a first tray onto a second tray, the first tray is rotated a predetermined amount relative to the second tray, and the interlock features on the edges of the first and second trays mate and are engaged. Male interlock features on one tray engage female interlock features on the other tray. Additional trays can be added to the stack in a similar manner: rotate the added tray and engage the interlock features on the added tray with the interlock features of the previous tray. Any number of trays can be added to the stack in this manner. Removing a tray from the stack of trays means simply disengaging the interlock features of the tray to be removed from the interlock features of the adjacent tray. The trays can be repeatedly added and removed from a stack without damaging the trays or the interlock features.

Certain examples are disclosed herein in the context of surgical instruments or tools used with cardiac treatments and other procedures. However, although certain principles disclosed herein may be particularly applicable to such procedures, it should be understood that the packaging systems, devices, and methods of the present disclosure may be used to package any suitable or desirable instruments, tools, components, and the like. Furthermore, examples of the present disclosure may be utilized with items in non-biological environments as well.

### Example Stackable Tray

As referenced above, the disclosed techniques, devices, and systems relate to packaging, and specifically to sterile packaging which can be advantageously used for storing surgical instruments or other items intended to be used in a sterile surgical field, for example. Disclosed packaging includes an example sterile stackable tray 102 that can be formed to enclose a single item or multiple items as desired. The disclosed concepts may be adapted to various packaging applications, including where the desire for sterile barrier management and a minimal footprint are regarded.

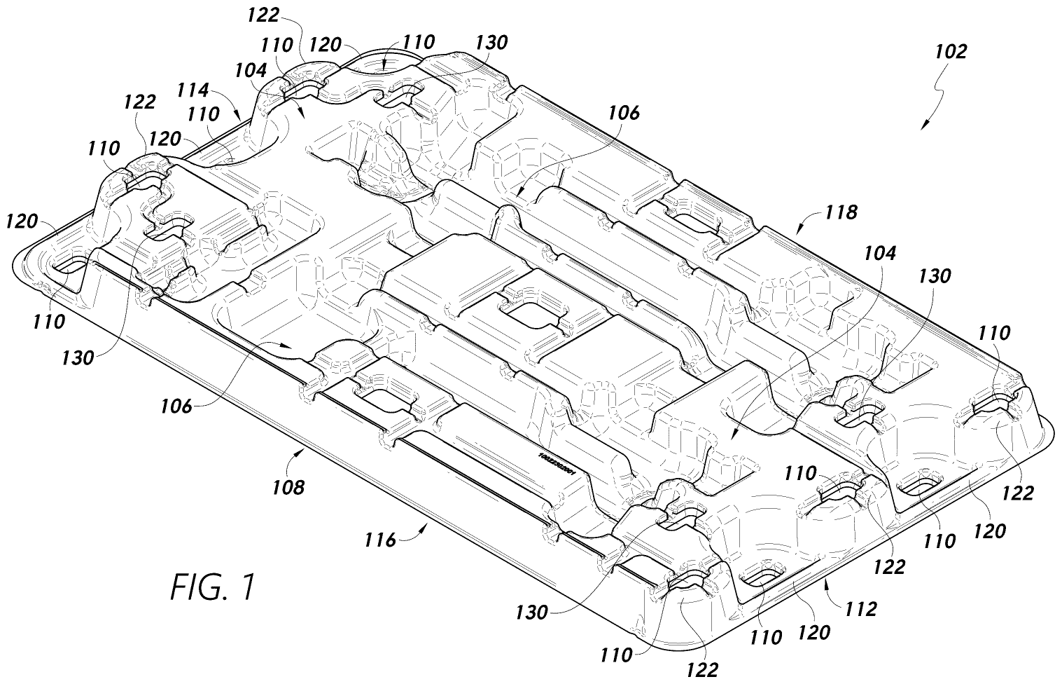

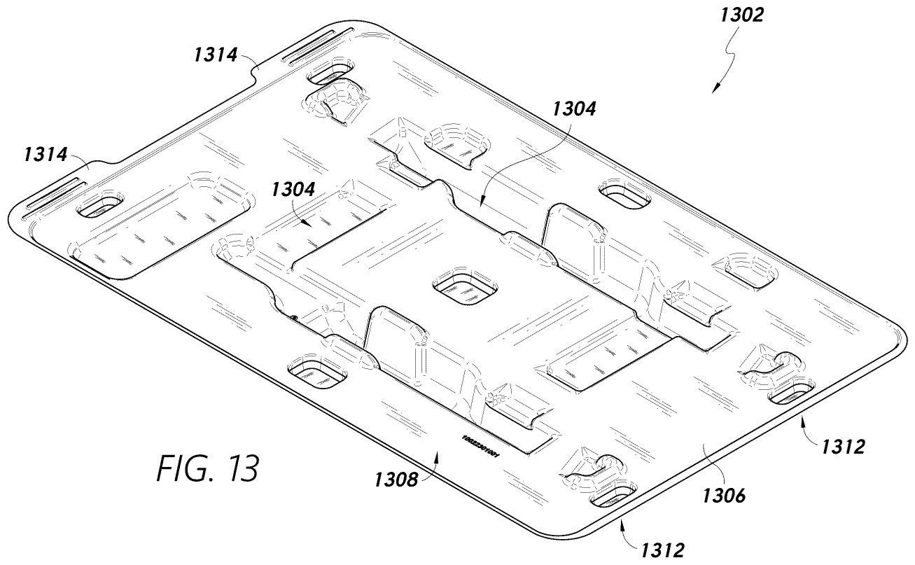

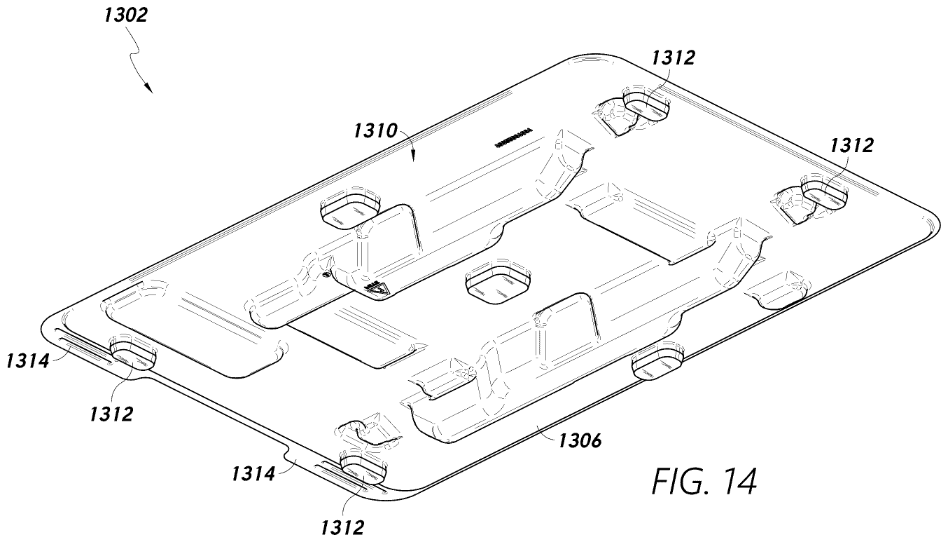

Referring to FIGS. 1-5, an example stackable tray 102 for packaging one or more implements is illustrated. Each stackable tray 102 comprises a formed lower portion or “tray” 102 which can be used with a re-sealable cover 1302 (see FIGS. 13-16). The tray 102. which may be comprised of a single layer of material or multiple (e.g., laminated) layers of material, can be formed (e.g., thermoformed) to accept one or more products and/or accessories. For instance, the tray 102 can be formed to include depressions 106 in the shapes of the items to be packaged, so as to provide protection to the enclosed items from damage as well as to keep them contaminate free. The cover 1302 can be formed to cover over desired items enclosed in the tray 102 and may also include formed features 1304 that conform to portions of the enclosed items. In various examples, the stackable tray 102 and the cover 1302 are sterile or sterilized and are configured to retain a sterility of sterile surgical instruments stored therein.

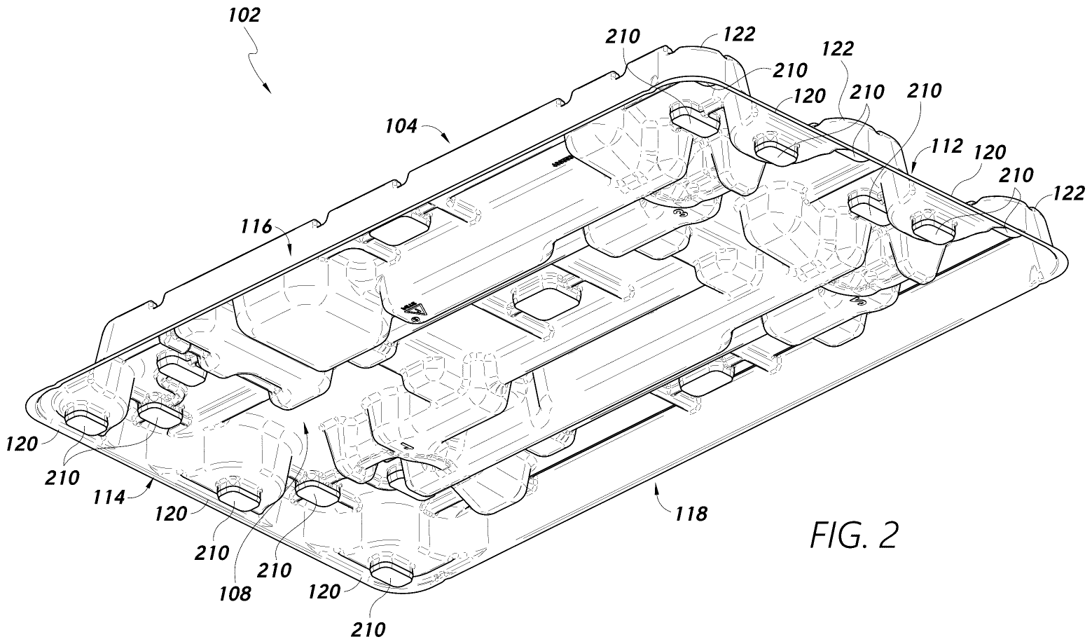

As shown at FIGS. 1 and 2, the stackable tray 102 includes a first surface 104 (e.g., “top” surface) configured to receive the one or more implements to be packaged and a second surface 108 (e.g., “bottom” surface) opposite the first surface 104. The first surface 104 of the stackable tray 102 can include at least one molded cavity 106 shaped to accommodate the one or more implements to be stored therein. A molded cavity 106 at the first surface 104 can be shaped to conform to at least a portion of the implement(s) to be packaged, or alternately the molded cavity 106 can have a more generic shape to accommodate various items that can be packaged at the stackable tray 102. The first surface 104 can include one molded cavity 106 or multiple molded cavities 106 for receiving one or more items to be packaged.

FIG. 1 illustrates a perspective view of an example stackable tray 102, with the topside surface (i.e., first surface 104) exposed. Referring to FIG. 1, the stackable tray 102 includes one or more first interlocking features 110 at the first surface 104 of the stackable tray 102. In some examples, the one or more first interlocking features 110 are disposed at a first side edge 112 of the stackable tray 102. Alternately or additionally, one or more first interlocking features 110 can be disposed at another side edge of the stackable tray 102, such as a second side edge 114 of the stackable tray 102, opposite the first side edge 112, or at one or more additional side edges 116, 118, etc. (based on the number of side edges of the stackable tray 102). In some implementations, one or more first interlocking features 110 are disposed at each of the first side edge 112 and the second side edge 114 of the first surface 104. Further, in some examples, one or more first interlocking features 110 may be disposed at interior areas of the first surface 104 of the first stackable tray 102, for example at an area within a predefined portion of the first surface 104. Alternately or additionally, one or more first interlocking features 110 can be disposed at the second surface 108 of the stackable tray 102.

FIG. 2 illustrates a perspective view of an example stackable tray 102, with the underside surface (i.e., second surface 108) exposed. Referring to FIG. 2, the stackable tray 102 includes one or more second interlocking features 210 at the second surface 108 of the stackable tray 102. In some examples, the one or more second interlocking features 210 are disposed at the second side edge 114 of the stackable tray 102. Alternately or additionally, one or more second interlocking features 210 can be disposed at another side edge of the stackable tray 102, such as the first side edge 112 of the stackable tray 102, opposite the second side edge 114, or at one or more additional side edges 116, 118, etc. (based on the number of side edges of the stackable tray 102). In some implementations, one or more second interlocking features 210 are disposed at each of the first side edge 112 and the second side edge 114 of the second surface 108. Further, in some examples, one or more second interlocking features 210 may be disposed at interior areas of the second surface 108 of the first stackable tray 102, for example at an area within a predefined portion of the second surface 108. Alternately or additionally, one or more second interlocking features 210 can be disposed at the first surface 104 of the stackable tray 102.

In various implementations, an example stackable tray 102 can have three or more side edges. In the examples shown in the figures (see FIGS. 1 and 2 for example), the stackable tray 102 has four side edges. For instance, the stackable tray 102 is illustrated as having a generally rectangular shape (in a plan view) and has two sets of opposing side edges: side edges 112 and 114 are one set of opposing side edges and side edges 116 and 118 are another set of opposing side edges. In other examples, a stackable tray 102 may have more than four side edges. In some examples, the stackable tray 102 may have at least one set of opposing side edges. In other examples, the stackable tray 102 may not have any opposing side edges. Further, in various examples, two or more side edges of a stackable tray 102 include one or more first 110 and/or second interlocking features, and in some examples each side edge of a stackable tray 102 includes at least one first 110 and/or second interlocking feature.

In an alternate implementation, an example stackable tray 102 has a generally circular or elliptical shape (in a plan view) and thus has a single side edge. Other irregular shapes are also conceivable where an example stackable tray 102 has a single side edge. In these examples, reference to a first side edge 112 and a second side edge 114 in the disclosure herein refer to locations on the single side edge, rather than separate side edges (as define a polygon, for example). In the examples, the referenced first side edge 112 and second side edge 114 locations may be adjacent, opposing, or have another relative spacing on the stackable tray 102.

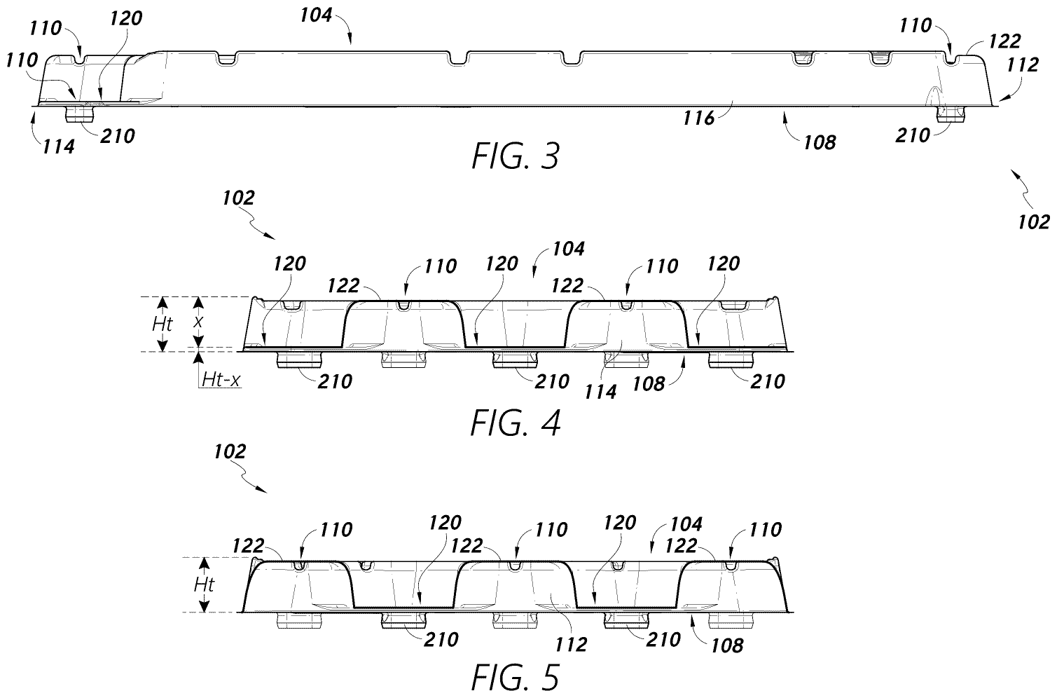

As shown at FIGS. 3-5, in an implementation, the stackable tray 102 includes first interlocking features 110 and second interlocking features 210 at each of the first 112 and second 114 side edges. Second interlock features 210 (i.e., “male” interlock features) can be disposed on one or more of the underside surfaces of the stackable tray 102 (protruding below the stackable tray 102) and first interlock features 110 (i.e., “female” interlock features) can be disposed on one or more of the topside surfaces of the stackable tray 102 (as recesses in the top surfaces of the tray). For example, first interlocking features 110 can be disposed at the first surface 104 and second interlocking features 210 can be disposed at the second surface 108. In an example, each first interlocking feature 110 is collinear with a second interlocking feature 210 on a line parallel to the axis 702 perpendicular to the plane of the stackable tray 102.

In various implementations, the stackable tray 102 is arranged to mate with another stackable tray via engagement of the second interlocking features 210 of one stackable tray 102 to the first interlocking features 110 of the other stackable tray 102. For instance, the second interlocking features 210 of a first stackable tray 102 are configured to be inserted into the first interlocking features 110 of a second stackable tray 102 to engage the second interlocking features 210 to the first interlocking features 110, which releasably couples the first stackable tray 102 to the second stackable tray 102.

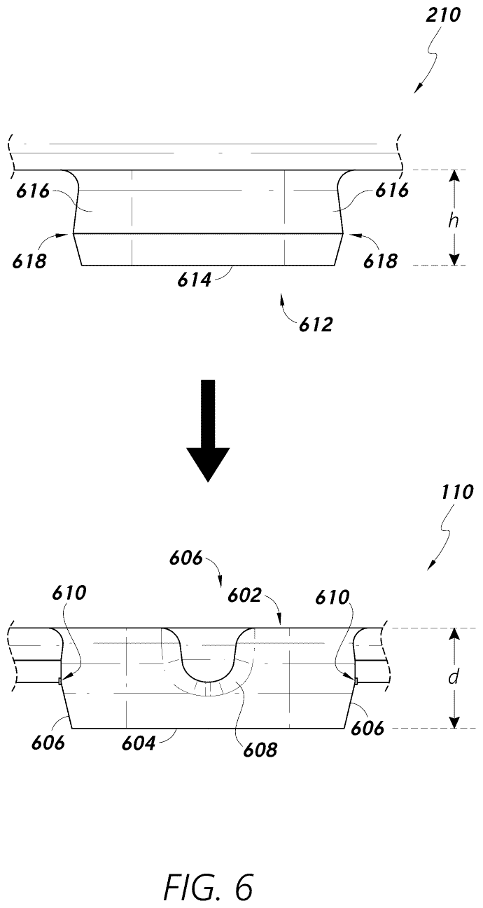

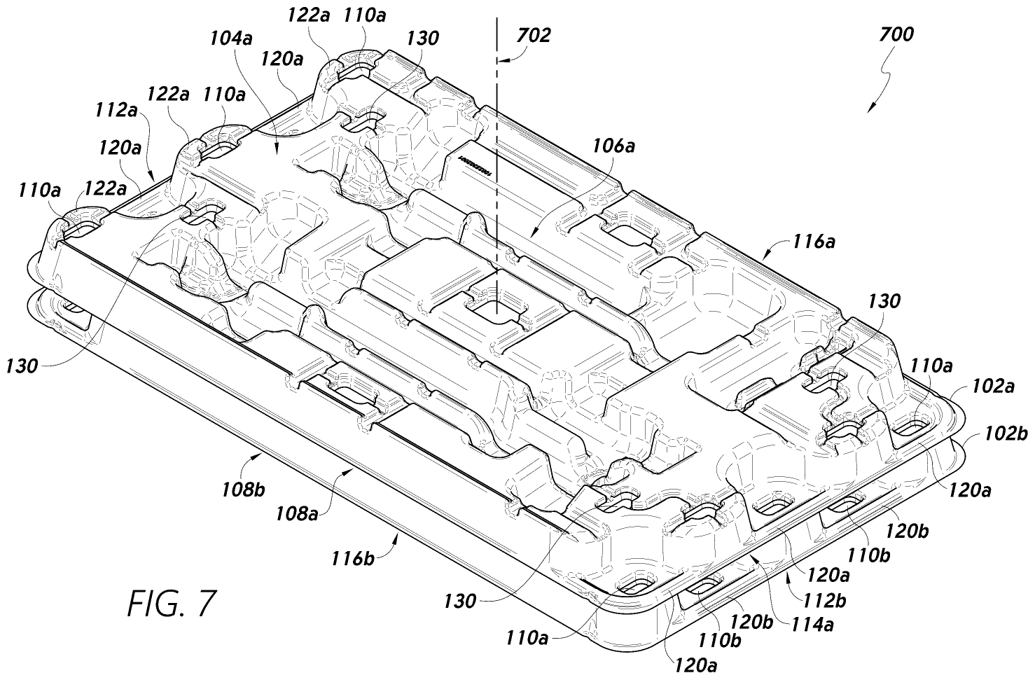

Referring to FIG. 6, detailed illustrations of example first 110 and second 210 interlocking features are shown in a profile view. The detailed illustrations of the example first 110 and second 210 interlocking features are shown by way of example for discussion purposes and are not intended to be limiting. In various implementations, first 110 and second 210 interlocking features can have other shapes and configurations. The first interlocking feature 110 is shown arranged to receive the second interlocking feature 210 for engagement. One or more of each of the first 110 and second 210 interlocking features may be disposed at stackable trays 102 as discussed for engaging the stackable trays 102 in a stack 700 (see FIG. 7, for example).

In various implementations, the one or more first interlocking features 110 each comprises a cavity 600 having an open end 602, a floor 604, and a peripheral side wall 606 defining a depth “d” of the cavity 600. In various examples, the cavity 600 has an elliptical shape, a polygonal shape, an irregular shape, or other shape in a plan view. The open end 602 is sized and shaped to receive the second interlocking feature 210. The floor 604 of the cavity 600 may be generally planar (as shown in the illustration of FIG. 6) or the floor 604 may have a non-planar topography. For example, the floor 604 may be partially or fully convex, concave, stepped. irregular, combinations of the above, and so forth.

The peripheral wall 606 of the cavity 600 defines the depth “d” of the cavity 600 and, along with the floor 604, defines the general interior shape of the cavity 600. The peripheral wall 606 may be continuous in surrounding the open end 602 and/or the floor 604. Alternately, the peripheral wall 606 may be discontinuous, e.g., have openings or breaks in the peripheral wall 606. For example, as shown at FIG. 6, the peripheral wall 606 may include a full or partial opening 608 along the length of the peripheral wall 606. The opening 608 may provide one or more various functions relative to the stackable tray 102, such as providing stress relief, flexibility, access, venting, and so forth to the cavity 600. For example, the presence of the opening 608 may improve the actions of engagement and disengagement of the first 110 and second 210 interlocking features, and thus the engagement and disengagement of the stackable trays 102. Further, the opening 608 may be implemented as part of the process of forming of the stackable tray 102 and may be advantageous to the formation process.

In various implementations, the second interlocking features 210 are configured to be inserted into the first interlocking features 110, and releasably engaged therein. In some implementations, the peripheral wall 606 of the cavity 600 includes one or more engaging features 610 arranged to provide a positive and secure engagement with the second interlocking feature 210. In various examples, the engaging features 610 may include one or more ridges, grooves, tabs, notches, bumps, recesses, dimples, and so forth. In an example, as shown at FIG. 6, the peripheral wall 606 of the cavity 600 includes an engaging feature 610 comprising a concave profile or recess in the surface of the peripheral wall 606 along at least a part of the peripheral wall 606. In the example, the concave engaging feature 610 of the peripheral wall 606 can define a slightly more narrow passage initially into the cavity 600 (e.g., near the cavity opening 602) above a wider portion of the cavity 600 (e.g., into a deeper part of the cavity 600), such that a predefined force is needed to insert the second interlocking feature 210 past the slightly more narrow passage of the engaging feature 610. Once past the narrower portion of the engaging feature 610, the second interlocking feature 210 is arranged to fit within the cavity 600 (including the wider portion of the engaging feature 610) of the first interlocking feature 110.

In various implementations, the one or more second interlocking features 210 each comprises a protrusion 612 having a maximal surface 614 and a peripheral side wall 616 defining a height “h” of the protrusion 612. In some examples, the protrusion 612 has an elliptical shape, a polygonal shape, an irregular shape, or other shape in a plan view. The protrusion 612 is sized and shaped to be received within the first interlocking feature 110. The maximal surface 614 comprises the bottom surface or extent of the protrusion 612. The maximal surface 614 may be generally planar (as shown in the illustration of FIG. 6) or the maximal surface 614 may have a non-planar topography. For example, the maximal surface 614 may be partially or fully convex, concave, stepped, irregular, combinations of the above, and so forth.

The peripheral side wall 616 of the protrusion 612 defines the height “h” of the protrusion 612 and, along with the maximal surface 614, defines the general exterior shape of the protrusion 612. The peripheral side wall 616 may comprise a continuous wall surrounding the maximal surface 614. Alternately, the peripheral side wall 616 may have one or more discontinuities, e.g., openings or breaks in the peripheral side wall 616.

In some implementations, the peripheral side wall 616 of the protrusion 612 includes one or more engaging features 618 arranged to provide a positive and secure engagement with the first interlocking feature 110 and/or including with the one or more engaging features 610 of the cavity 600. In various examples, the engaging features 618 may include one or more ridges, grooves, tabs, notches, bumps, recesses, dimples, and so forth. In an example, as shown at FIG. 6, the peripheral side wall 616 of the protrusion 612 includes an engaging feature 618 comprising a convex profile or bulge in the surface of the peripheral side wall 616 along at least a part of the peripheral side wall 616. In the example, the convex engaging feature 618 of the peripheral side wall 616 can define a slightly wider profile portion of the protrusion 612, such that a predefined force is needed to insert the second interlocking feature 210 into the first interlocking feature 110. Once past the slightly wider portion of the engaging feature 618, the second interlocking feature 210 is arranged to fit within the cavity 600 of the first interlocking feature 110. In an example, the shape of the engaging feature 618 of the protrusion 612 is arranged to conform to the shape of the engaging feature 610 of the cavity 600.

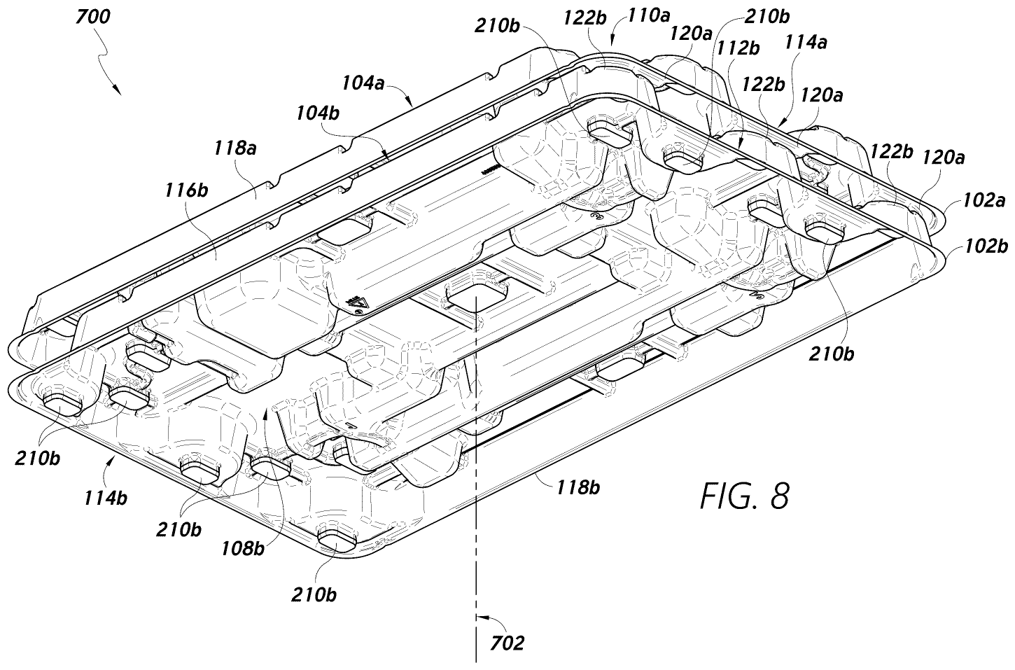

In various implementations, the stackable tray 102 is arranged to mate with another stackable tray via engagement of the second interlocking features 210 of one stackable tray 102 to the first interlocking features 110 of the other stackable tray 102 when the first stackable tray 102 is rotated a predetermined amount about an axis perpendicular to a plane of the first stackable tray 102 (i.e., the axis 702 illustrated at FIGS. 7-10), from a first orientation to a second orientation, relative to the other stackable tray 102. For instance, referring to FIGS. 7-10, the second interlocking features 210 of the first stackable tray 102 are aligned to be inserted into the first interlocking features 110 of the second stackable tray 102, to engage the second interlocking features 210 to the first interlocking features 110, when the first stackable tray 102 is rotated the predetermined rotational amount relative to the second stackable tray 102. In other words, rotating the first stackable tray 102 the predetermined amount relative to the second stackable tray 102 aligns the second interlocking features 210 of one stackable tray 102 to the first interlocking features 110 of the other stackable tray 102 for engagement. In some cases, when the first stackable tray 102 is not rotated relative to the second stackable tray 102 (i.e., the first and second stackable trays 102 have the same orientation), the second interlocking features 210 of one stackable tray 102 may not be aligned to first interlocking features 110 of the other stackable tray 102 for engagement. In an alternate implementation, the second interlocking features 210 of one stackable tray 102 are aligned to first interlocking features 110 of the other stackable tray 102 for engagement when the first and second stackable trays 102 have the same orientation and when the first stackable tray 102 is rotated the predetermined amount relative to the second stackable tray 102.

In various implementations, the predetermined rotational amount is greater than 0 degrees and less than 360 degrees. In an example with generally rectangular-shaped stackable trays 102 (as shown in the figures), the predetermined rotational amount can be one or more of 90 degrees, 180 degrees, or 270 degrees, depending on the arrangement of the first 110 and second 210 interlocking features on the stackable trays 102. These rotations can allow for the first 110 and second 210 interlocking features on the respective stackable trays 102 to align and can also preserve a minimal footprint of the stack 700, which can comprise the footprint of a single stackable tray 102. In examples of stackable trays with more than four sides, additional or alternate predetermined rotational amounts are contemplated, such that sides of the respective stackable trays 102 are aligned (to preserve the footprint of a single stackable tray 102) and to align second interlocking features 210 of one stackable tray 102 to first interlocking features 110 of another stackable tray 102 for engagement. For example, in the case of a five-sided stackable tray 102, the predetermined rotational amount can include one or more of 72 degrees. 144 degrees. 216 degrees, and 288 degrees, depending on the arrangement of the first 110 and second 210 interlocking features on the stackable trays 102. As a person having skill in the art will appreciate. any number of like stackable trays 102 can be stacked into a stack 700, with each subsequent stackable tray 102 added to the stack 700 being rotated the predetermined amount, relative to the stackable tray 102 to be engaged.

In some examples, the stackable trays 102 in the stack 700 are identical. For example, referring to FIGS. 7-10, examples of identical stackable trays 102a and 102b are shown for illustrative purposes. In the example, the first stackable tray 102a is rotated about the perpendicular axis 702 relative to the second stackable tray 102b. In the example, the predetermined rotational amount is 180 degrees. Accordingly, the first stackable tray 102a is rotated 180 degrees relative to the second stackable tray 102b, which aligns the first side edge 112b of the second stackable tray 102b to the second side edge 114a of the first stackable tray 102a and the first side edge 112a of the first stackable tray 102a to the second side edge 114b of the second stackable tray 102b.

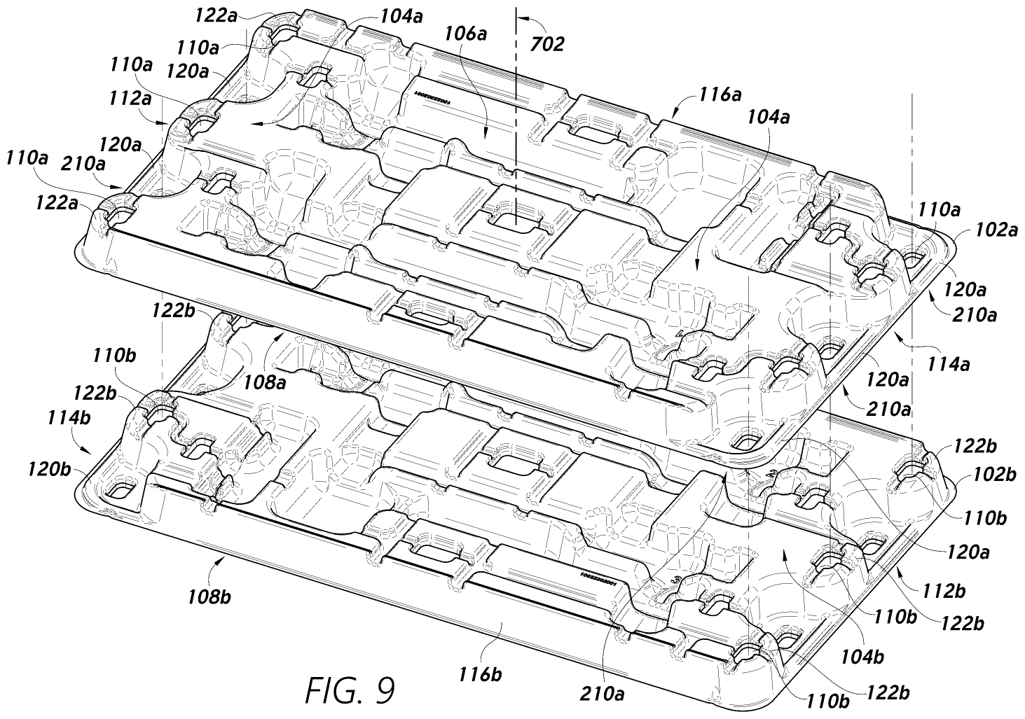

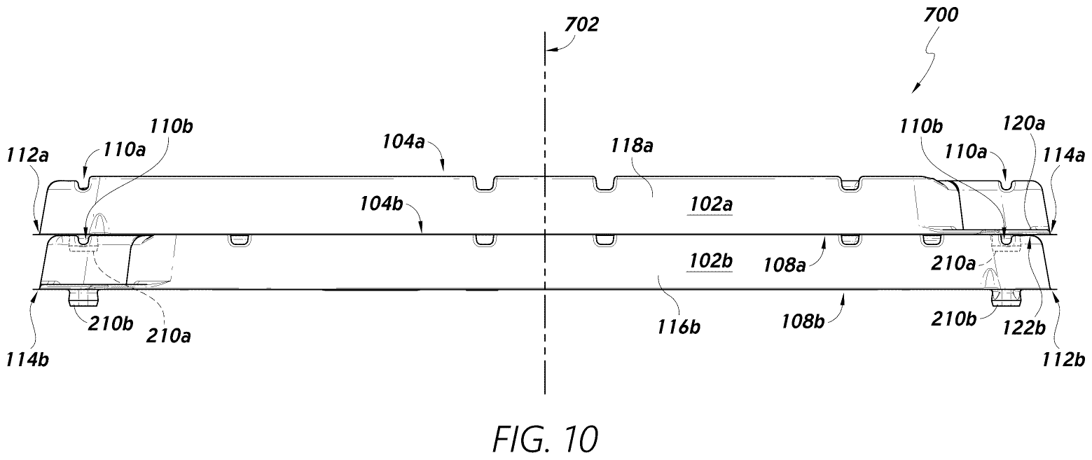

As shown at FIGS. 9 and 10, this relative rotation also aligns the second interlocking features 210a of the first stackable tray 102a with the first interlocking features 110b of the second stackable tray 102b for engagement. As shown in the figures, inserting and engaging the second interlocking features 210a of the first stackable tray 102a with the first interlocking features 110b of the second stackable tray 102b releasably engages the first stackable tray 102a to the second stackable tray 102b, forming the stack 700. In the example, a third stackable tray 102 (not shown) can be stacked to the first 102a or second 102b stackable trays by rotating the third stackable tray 102 the predetermined rotational amount (e.g., 180 degrees) relative to the first stackable tray 102a and engaging the third stackable tray 102 to the first stackable tray 102a or rotating the third stackable tray 102 the predetermined rotational amount (e.g., 180 degrees) relative to the second stackable tray 102b and engaging the third stackable tray 102 to the second stackable tray 102b, either of which increases the quantity of stackable trays 102 in the stack 700. Any number of additional stackable trays 102 can be added to the stack 700 in like manner.

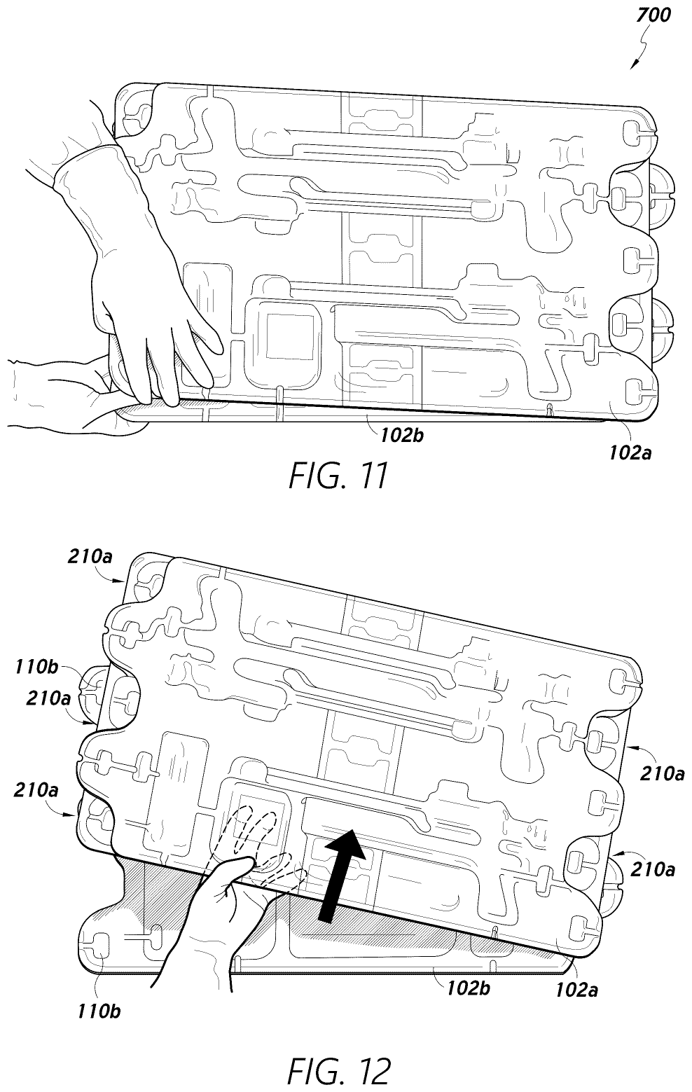

Referring to FIGS. 11 and 12, the stack 700 can be separated into individual trays 102a and 102b as desired. As shown, the stackable tray 102a is lifted away from the stackable tray 102b, disengaging the second interconnect features 210a of the first stackable tray 102a from the first interconnect features 110b of the second stackable tray 102b. In an example, the stackable trays 102 can be re-engaged and disengaged as many times as desired, with the same or different stackable trays 102.

As shown at FIGS. 1-5 and 7-12. in various implementations one or more side edges of a stackable tray 102 are contoured. For instance, in an example as shown, the first 112 and second 114 side edges are contoured. In the example, at least one of the first side edge 112 and the second side edge 114 includes a recessed portion 120 that is recessed below the first surface 104. In other words, as shown at FIGS. 4 and 5 the stackable tray 102 (and the first surface 104) has a height “Ht” and the recessed portion 120 has a height that is less than the height “Ht” of the stackable tray 102 and the first surface 104. In the example, the recessed portion 120 has a height “Ht−x”, where “x” is some distance less than the distance “Ht”. Thus, the recessed portion 120 is recessed below the first surface 104 a distance “x”. In one example, the height “Ht” of the stackable tray 102 (and the first surface 104) is about 3 to 5 cm and the height “Ht−x” of the recessed portion 120 is about 0.2 to 0.5 cm. In alternate examples, the height of the stackable tray 102 (and the first surface 104) is less than 3 cm or greater than 5 cm, and the height of the recessed portion 120 is less than 0.2 cm or greater than 0.5 cm. As shown at FIG. 1, in various examples, one of the one or more first interlocking features 110 is disposed at the recessed portion 120.

As shown at FIGS. 1-5 and 7-12, in various implementations the recessed portion 120 is flanked by a raised portion 122 at the first side edge 112 and/or the second side edge 114. The raised portion 122 is elevated above the recessed portion 120. In other words, as shown at FIGS. 4 and 5, the raised portion 122 has a height greater than the height “Ht−x” of the recessed portion 120. In some implementations, the raised portion 122 has a height “Ht” or a height of the stackable tray 102 and is level with the first surface 104. As shown at FIG. 1, in various examples, one of the one or more first interlocking features 110 is disposed at the raised portion 122.

In various implementations, the recessed portion 120 is flanked by a pair of raised portions 122 at the first side edge 112 and/or the second side edge 114. A raised portion 122 is disposed at each flank of the recessed portion 120, with each raised portion 122 being elevated above the recessed portion 120. In various examples, a first interlocking feature 110 is disposed at each raised portion 122. As illustrated at FIGS. 1-5 and 7-12, in various implementations each raised portion 122 is bordered by a subsequent recessed portion 120 that is recessed below the first surface 104. Each subsequent recessed portion 120 can have a first interlocking feature 110 disposed thereat. For example, one or both of the first side edge 112 and the second side edge 114 of the stackable tray 102 includes at least two recessed portions 120 and at least two raised portions 122, with each recessed portion 120 being recessed below the first surface 104 and each raised portion 122 being elevated above the recessed portions 120. A first interlocking feature 110 can be disposed at each of the at least two recessed portions 120 and the at least two raised portions 122.

Referring to FIGS. 1-5 and 7-12, in various implementations, a pattern of the at least two recessed portions 120 and the at least two raised portions 122 at the first side edge 112 and the second side edge 114 of the respective first 102a and second 102b stackable trays comprises an alternating pattern of the at least two recessed portions 120 and the at least two raised portions 122. In alternate implementations, a pattern of the at least two recessed portions 120 and the at least two raised portions 122 at the first side edge 112 and the second side edge 114 of the respective first 102a and second 102b stackable trays can include two or more adjacent recessed portions 120 and/or two or more adjacent raised portions 122.

In various implementations, the pattern of the at least two recessed portions 120 and the at least two raised portions 122 at the first side edge 112 of the stackable tray 102 comprises an inverse pattern of the at least two recessed portions 120 and the at least two raised portions 122 at the second side edge 114 of the stackable tray 102. In other words, the pattern of the first side edge 112 is an inverse to the pattern of the second side edge 114. As shown at FIGS. 7-12, the inverse side edge pattern facilitates engagement of the first 110 and second 210 interlocking features of two stackable trays 102 when one stackable tray 102 is rotated a predetermined rotational amount relative to another stackable tray 102. For example, referring to FIG. 9, the first stackable tray 102a is rotated 180 degrees relative to the second stackable tray 102b, which aligns the second interlocking features 210a of the first stackable tray 102a to the first interlocking features 110b of the second stackable tray 102b. Additionally, the rotation of the first stackable tray 102a aligns the recessed portions 120a of the first stackable tray 102a to the raised portions 122b of the second stackable tray 102b, putting the second interlocking features 210a of the first stackable tray 102a in reach of the first interlocking features 110b of the second stackable tray 102b. With the second interlocking features 210a of the first stackable tray 102a in reach of the first interlocking features 110b of the second stackable tray 102b, the interlocking features can be engaged, which engages the first 102a and second 102b stackable trays, forming the stack 700 (see FIG. 7). Thus, the recessed portions 120 of a first stackable tray 102a and the raised portions 122 of a second stackable tray 102b align in order to reach the respective interconnect features (110 and 210) for engagement.

In various implementations, the second stackable tray 102b is identical to the first stackable tray 102a, which allows the one or more first interlocking features 110b of the second stackable tray 102b to be aligned with and to resiliently engage the one or more second interlocking features 210a of the first stackable tray 102a when the first stackable tray 102a is rotated from a first orientation (at zero degrees, relative to the second stackable tray 102b) to a second orientation (at 180 degrees, for example, relative to the second stackable tray 102b) and the first stackable tray 102a is stacked onto the second stackable tray 102b. The second orientation comprises a predetermined rotational displacement that is based on the number of sides of the stackable trays 102 and the arrangement of the first 110 and second 210 interconnect features disposed thereon.

The first 110 and second 210 interlock features can be arranged in various configurations, including along or near the side edges of the stackable tray 102, so long as they are consistent on every stackable tray 102 (e.g., at least the arrangement of the first 110 and second 210 interlock features on each stackable tray 102 is identical), so that second interlock features 210 engage with first interlock features 110 when a first stackable tray 102 is rotated a predetermined rotational displacement relative to a second stackable tray 102. A first stackable tray 102 to be stacked with another stackable tray 102 can be either stacked above the other stackable tray 102 or below the other stackable tray 102. Other arrangements of the first 110 and second 210 interlock features than those described herein are contemplated, and will function in like manner so long as the first 110 and second 210 interlock features engage when a stackable tray 102 is rotated the predetermined rotational amount (e.g., 180 degrees) relative to an adjacent stackable tray 102. For instance, first 110 and second 210 interlock features can be disposed on additional side edges of a stackable tray 102 (alternate or additional to the first 112 and second 114 side edges) or at an interior portion of a stackable tray 102 in some examples.

The stackable tray 102 comprises a thermoformed (or the like) sheet (or multiple sheets) of a thermoplastic polymer, such as polyethylene terephthalate glycol (PETG), for one example. Polystyrene or other polymers may also be used, as well as various other composites or other materials. PETG is widely used in the medical fields due to its chemical resistance, durability, formability, impact and temperature resistance, and other properties. The addition of glycol, which modifies polyethylene terephthalate (PET) to become PETG, gives the added property of allowing multiple stackable trays 102 to be easily separated, among other characteristics.

The stackable tray 102 may be vacuum and pressure formed with a single die (or molded using a single mold) to produce multiple identical stackable trays 102. This results in less thermoforming tools, for reduced development and manufacturing processes and greater cost savings. The interlocking features 110 and 210 can be formed as part of the process of forming the stackable trays 102. In an example, the first interlocking feature 110 and the second interlocking feature 210 are formed together, via the die or mold, by deforming the sheet(s) of polymer at the various locations. This results in each first interlocking feature 110 being collinear with a second interlocking feature 210 on a line parallel to the axis 702 perpendicular to the plane of the stackable tray 102. The cover 1302 can also be formed from a thermoplastic polymer in a like manner, using a single mold to form multiple covers 1302.

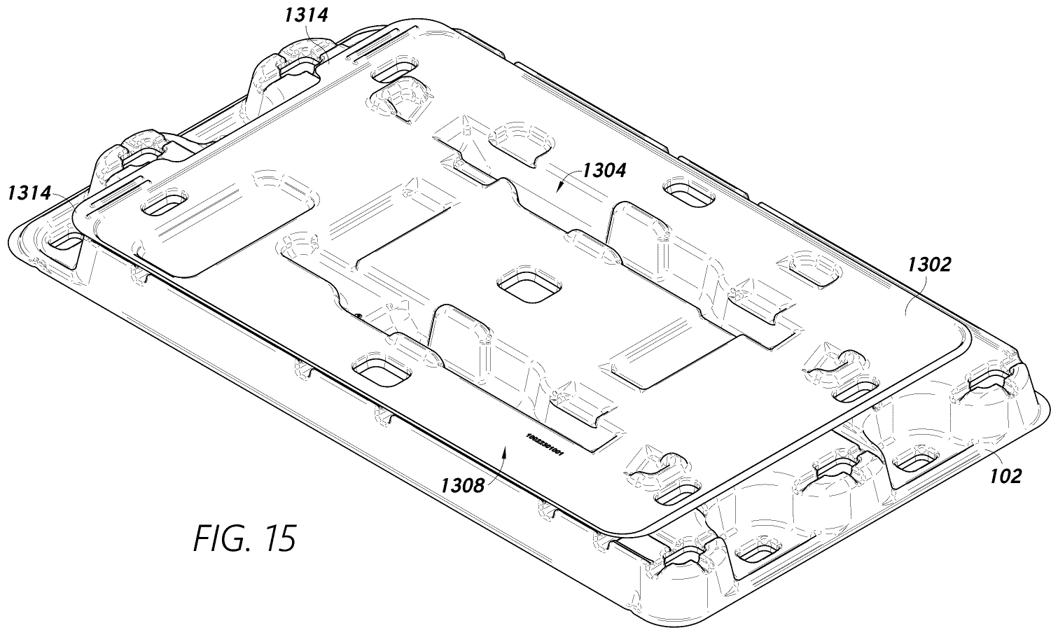

Referring to FIGS. 13-16, each stackable tray 102 can have a removable and replaceable sterile cover 1302. The cover 1302 comprises a substantially planar sheet 1306 with a first (top or upper surface) side 1308 and a second (bottom or lower surface) side 1310. The cover 1302 may also include formed features 1304 that conform to portions of the enclosed items. The cover 1302 includes molded (e.g., thermoformed) tabs 1312 that protrude from the lower surface 1310 of the cover 1302, similar to the male interlock features 210. For example, the material of the cover sheet 1306 can be pressed and deformed at various locations to result in tabs 1312 that protrude from the underside 1310 of the cover sheet 1306 at those locations. The molded tabs 1312 snap into molded recesses 130 (see FIG. 1, for example) in the first surface 104 of the stackable tray 102 to resiliently engage the molded recesses and secure the cover 1302 to the stackable tray 102, sealing the stackable tray 102. The corresponding tabs 1312 and recesses 130 may be located near the edges of the cover 1302 and at locations at the interior regions of the stackable tray 102 and the cover 1302. As shown at FIG. 15, the cover 1302 can be disposed inside a perimeter of the interlock features 110 and 210 of the stackable tray 102 so as not to interfere with the stacking and interlocking of the stackable tray 102. For example, the length and width of the cover 1302 fits within a perimeter defined by the first interlocking features 110 at the first surface 104 of the stackable tray 102.

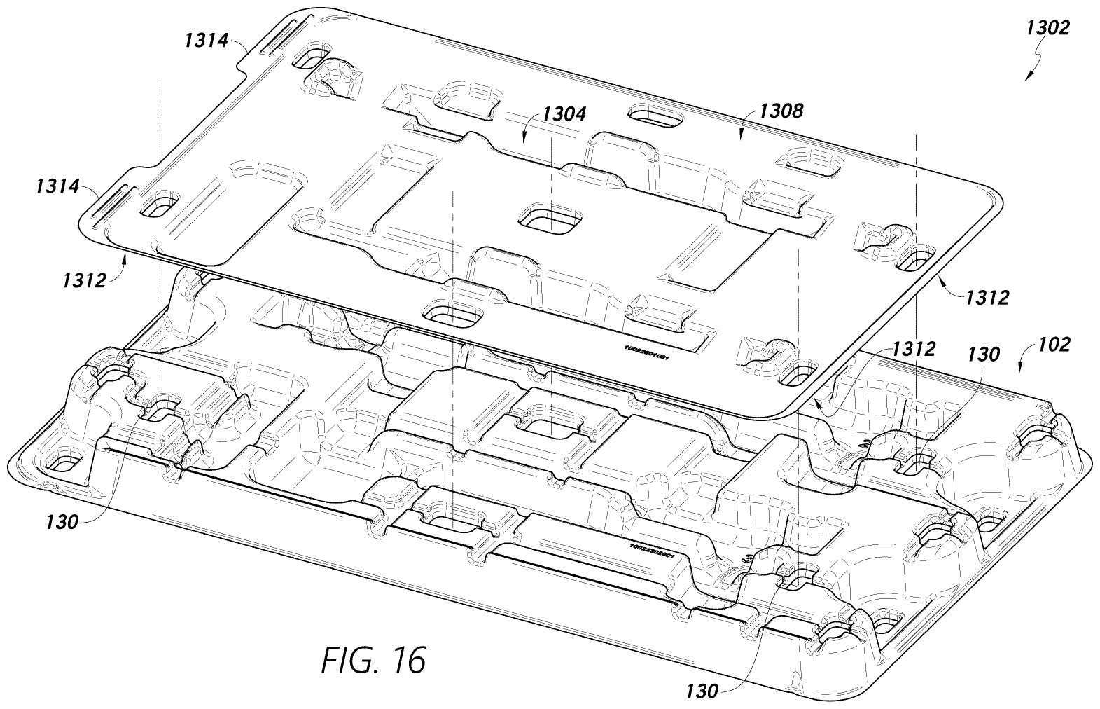

The cover 1302 can be removed by lifting the 1302 cover to release the tabs 1312 of the cover 1302 from the recesses 130 of the stackable tray 102. The cover 1302 may include one or more lifting tabs 1314 to assist in removing the cover 1302 from the stackable tray 102. The cover 1302 can be replaced or closed by locating the cover 1302 so that the tabs 1312 are aligned over the corresponding recesses 130 of the stackable tray 102, as shown at FIG. 16, and then pressing the tabs 1312 into the recesses 130 to snap them into the recesses 130. The stackable trays 102 can be stacked into the stack 700 as described above while the cover 1302 is in place on the stackable trays 102. In some examples, multiple stackable trays 102 in a stack 700 can be scaled in a sterile pouch, for example. In either case, a stack 700 of stackable trays 102 sealed with covers 1302 can be passed through the sterile barrier and into the surgical field at once.

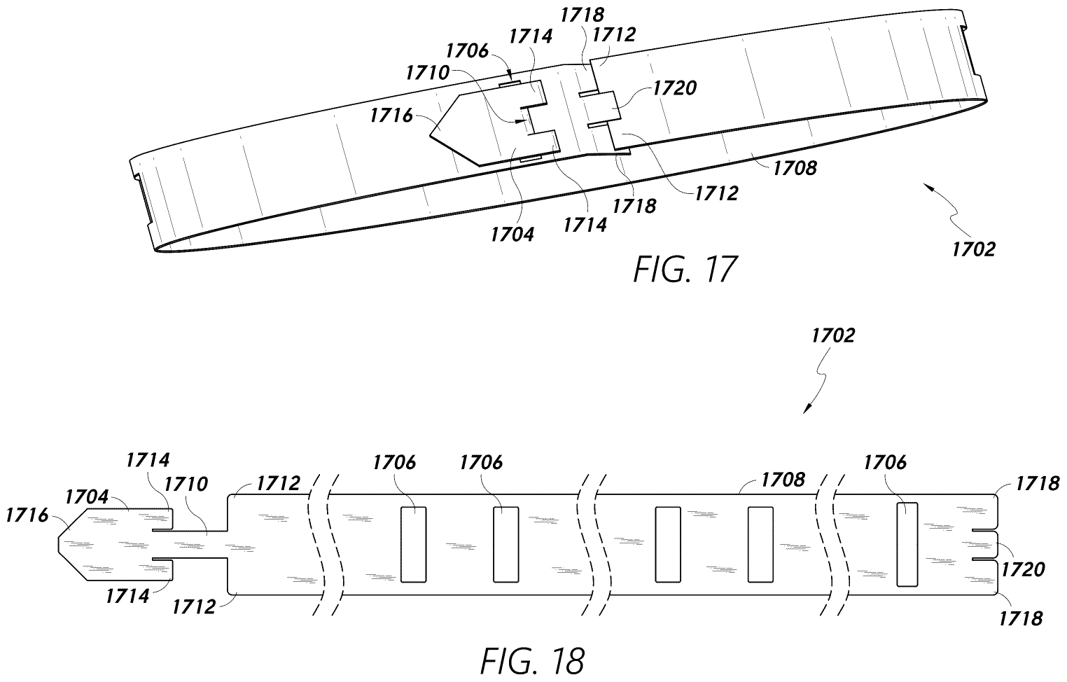

Referring to FIGS. 17 and 18, in some examples the stack 700 of stackable trays 102 can be held together using one or more novel belts 1702. The belt 1702 facilitates the handling of multiple stackable trays 102 by securing the stack 700 for storage or transport. The belt 1702 includes a locking mechanism comprising a latch 1704 and one or more slots 1706 disposed along at least a portion of the belt body 1708. The latch 1704 is either coupled to the belt body 1708 or can be integral to the belt body 1708 via a narrow neck 1710. The narrow neck 1710 defines a shoulder 1712 (or two shoulders 1712) at the first end of the belt body 1708, where the shoulder 1712 is a transition from the narrow width of the narrow neck 1710 to a wider width of the belt body 1708. In some examples, the shoulder 1712 is a gradual or diagonal transition and in other examples (as shown at FIGS. 17 and 18) the shoulder 1712 is an abrupt or step transition.

The latch 1704 is arranged to be inserted into a slot 1706 and releasably engaged to the slot 1706. For instance, the latch 1704 includes a front end 1716 that may be pointed, wedge-shaped, rounded, curved, blunted, or other shape. The shape of the front end 1716 can facilitate its insertion through a slot 1706. The latch 1704 also includes at least one rear tab 1714 where the latch 1704 meets the narrow neck 1710. In an example, as shown at FIGS. 17 and 18, the latch 1704 includes a rear tab 1714 on either side of the narrow neck 1710. When the latch 1704 is put through a slot 1706, the rear tab 1714 can prevent the latch 1704 from backing out of the slot 1706. The rear tab 1714 can engage the slot 1706 by extending out of the slot 1706 toward or along the belt body 1708 and away from the front end 1716.

With the latch 1704 engaged at the slot 1706, the belt 1702 can be further secured by engaging one or more locking tabs 1718 to the shoulder 1712 (or engaging two locking tabs 1718 to two shoulders 1712). The locking tab(s) 1718 are disposed at the second end of the belt body 1708. at the opposite end of the belt body 1708 from the latch 1704. The locking tabs 1718 can comprise portions of the second end of the belt body 1708 formed by longitudinal cuts at the second end of the belt body 1708, as shown at FIG. 18. The locking tabs 1718 may be separated by a center tab 1720. The locking tabs 1718 can be engaged to the shoulder(s) 1712 by extending the locking tabs 1718 beneath the shoulder(s) 1712 while the center tab 1720 is disposed above the narrow neck 1710 and the belt body 1708. By such engagement, the latch 1704 is further secured in place until intentionally removed.

The latch 1704 may be intentionally removed to release the belt 1702 by disengaging the locking tabs 1718 from the shoulders 1712 and disengaging the rear tabs 1714 from the slot 1706. The latch 1704 can then be backed out of the slot 1706, which releases the belt 1702. In some implementations, the belt 1702 can be torn or cut to release the belt 1702 from the stack 700. For instance, the belt 1702 may be manufactured from a paper product or a mix of materials that may include paper or textiles, such that the belt 1702 has sufficient strength but is capable of being torn or cut. In other examples, the belt 1702 can be manufactured from a polymer, a composite, a combination, or like materials. In an implementation, the belt 1702 is manufactured to be a single integral component with the latch 1704 and the narrow neck 1710. Alternately, the latch 1704 and/or the narrow neck 1710 may be manufactured separately and coupled to the belt body 1708.

### Example Process

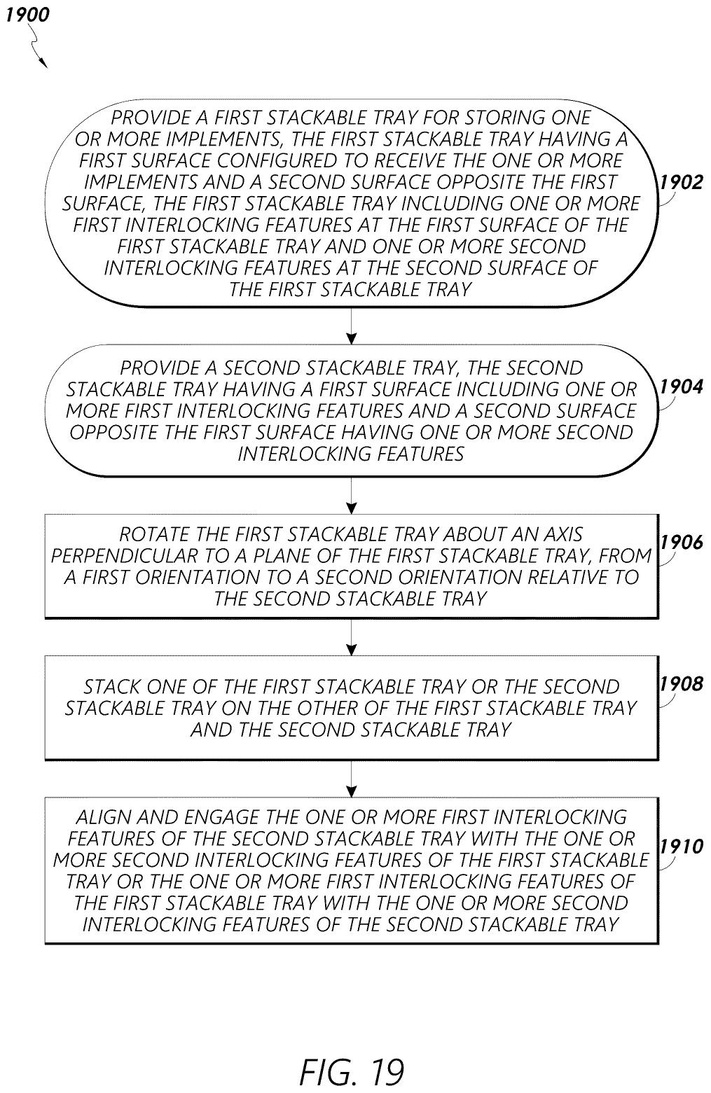

Referring to FIG. 19, a flow diagram illustrates an example process 1900 for stacking stackable trays (such as stackable trays 102, for example, as shown at FIGS. 1-12.) One or more of the process steps can be removed or adjusted while remaining within the scope of the disclosure. Further, the order of the process steps is not intended to be limiting, unless specifically indicated.

At block 1902, the process includes providing a first stackable tray for storing one or more implements. The first stackable tray has a first surface configured to receive the one or more implements and a second surface opposite the first surface. The first stackable tray includes one or more first interlocking features at the first surface of the first stackable tray and one or more second interlocking features at the second surface of the first stackable tray. In an example, the first surface of the first stackable tray includes at least one molded cavity shaped to accommodate the one or more implements.

At block 1904, the process includes providing a second stackable tray. The second stackable tray has a first surface including one or more first interlocking features and a second surface opposite the first surface having one or more second interlocking features. In an example, the second stackable tray is identical to the first stackable tray. The first stackable tray and the second stackable tray are sterile or sterilized and are configured to retain a sterility of sterile surgical instruments stored therein.

In an implementation, the one or more first interlocking features of the first and second stackable trays each comprises a cavity having an open end, a floor, and a peripheral side wall defining a depth of the cavity. Further, the one or more second interlocking features of the first and second stackable trays each comprises a protrusion having a maximal surface and a peripheral side wall defining a height of the protrusion. In an example, each first interlocking feature of the one or more first interlocking features is collinear with a second interlocking feature of the one or more second interlocking features on a line parallel to the axis perpendicular to the plane of the first stackable tray.

In an implementation, each of the first and second stackable trays includes a first side edge and a second side edge. In an example, the first and second side edges of the respective first and second stackable trays are opposing side edges. The one or more first interlocking features are disposed at the first side edge and the second side edge of the first and second stackable trays and the one or more second interlocking features are disposed at the first side edge and the second side edge of the first and second stackable trays. In an example, the first side edge and the second side edge of each of the first and second stackable trays includes at least two recessed portions and at least two raised portions. Each recessed portion is recessed below the first surface of the respective first and second stackable trays and each raised portion is elevated above the recessed portions. A first interlocking feature of the one or more first interlocking features of the first and second stackable trays is disposed at each of the at least two recessed portions and the at least two raised portions of the first and second stackable trays.

In an implementation, a pattern of the at least two recessed portions and the at least two raised portions at the first side edge of the respective first and second stackable trays comprises an inverse pattern of the at least two recessed portions and the at least two raised portions at the second side edge of the respective first and second stackable trays. In an example, a pattern of the at least two recessed portions and the at least two raised portions at the first side edge of the respective first and second stackable trays comprises an alternating pattern of the at least two recessed portions and the at least two raised portions.

At block 1906, the process includes rotating the first stackable tray about an axis perpendicular to a plane of the first stackable tray, from a first orientation to a second orientation relative to the second stackable tray. Rotating the first stackable tray from the first orientation to the second orientation comprises rotating the first stackable tray by a predetermined rotational amount or rotational displacement. In one example, the predetermined rotational amount or rotational displacement comprises 90 degrees. In another example, the predetermined rotational amount or rotational displacement comprises 180 degrees. In other examples, the predetermined rotational amount or rotational displacement comprises between 0 and 360 degrees.

At block 1908, the process includes stacking one of the first stackable tray or the second stackable tray on the other of the first stackable tray and the second stackable tray.

At block 1910, the process includes aligning and engaging the one or more first interlocking features of the second stackable tray with the one or more second interlocking features of the first stackable tray or the one or more first interlocking features of the first stackable tray with the one or more second interlocking features of the second stackable tray. For example, aligning comprises mating a recessed portion at the first side edge of one of the first and second stackable trays to a raised portion at the second side edge of the other of the first and second stackable trays. Further, engaging comprises inserting the second interlocking features of the first stackable tray into the first interlocking features of the second stackable tray or inserting the second interlocking features of the second stackable tray into the first interlocking features of the first stackable tray.

In an implementation, the process includes sealing a rescalable cover to the first surface of the first stackable tray or the second stackable tray by resiliently engaging a plurality of first engagement features on a first surface of the cover to a plurality of second engagement features on the first surface of the respective first stackable tray or the second stackable tray.

In an implementation, the process includes securing the first stackable tray to the second stackable tray using a belt. The belt includes a belt body comprising a flat strap having one or more slots disposed thereon and a latch coupled to or integral with a first end of the belt body via a narrow neck. The latch has a front end and at least one rear tab and the narrow neck defines a shoulder at the first end of the belt body. In an example, the process includes wrapping the belt body around the first and second stackable trays, inserting the latch through one of the one or more slots, engaging the at least one rear tab of the latch to the one of the one or more slots to prevent the latch from backing out of the one of the one or more slots, and engaging a locking tab disposed at a second end of the belt body to the shoulder.

In alternate implementations, the belt may include additional or alternate components or elements, without departing from the scope of the disclosure.

### Additional Description of Examples

Provided below is a list of examples, each of which may include aspects of any of the other examples disclosed herein. Furthermore, aspects of any example described above may be implemented in any of the numbered examples provided below.

### Example 1

A packaging system comprising: a first stackable tray for storing one or more implements, the first stackable tray having a first surface configured to receive the one or more implements and a second surface opposite the first surface, the first stackable tray including: one or more first interlocking features at the first surface of the first stackable tray; and one or more second interlocking features at the second surface of the first stackable tray, the first stackable tray being arranged to mate with a second stackable tray via engagement of the first interlocking features or the second interlocking features when the first stackable tray is rotated about an axis perpendicular to a plane of the first stackable tray, from a first orientation to a second orientation, relative to the second stackable tray.

### Example 2

The packaging system of any example herein, in particular example 1, further comprising a first side edge of the first stackable tray and a second side edge of the first stackable tray, wherein at least one of the one or more first interlocking features is disposed at the first side edge and at least one of the one or more second interlocking features is disposed at the second side edge.

### Example 3

The packaging system of any example herein, in particular example 2, wherein at least one other of the one or more first interlocking features is disposed at the second side edge and at least one other of the one or more second interlocking features is disposed at the first side edge.

### Example 4

The packaging system of any example herein, in particular any of examples 2-3, wherein the second stackable tray is identical to the first stackable tray, and wherein one or more second interlocking features of the second stackable tray are arranged to be aligned with and to engage the one or more first interlocking features of the first stackable tray when when the first stackable tray is rotated from the first orientation to the second orientation and the second stackable tray is stacked on the first stackable tray.

### Example 5

The packaging system of any example herein, in particular example 4, wherein rotating the first stackable tray from the first orientation to the second orientation comprises rotating the first stackable tray 90 degrees from the first orientation.

### Example 6

The packaging system of any example herein, in particular example 4, wherein rotating the first stackable tray from the first orientation to the second orientation comprises rotating the first stackable tray 180 degrees from the first orientation.

### Example 7

The packaging system of any example herein, in particular any of examples 2-6, wherein the first and second side edges are opposing side edges.

### Example 8

The packaging system of any example herein, in particular any of examples 2-6, wherein the first side edge includes a recessed portion that is recessed below the first surface, and wherein one of the one or more first interlocking features is disposed at the recessed portion.

### Example 9

The packaging system of any example herein, in particular example 8, wherein the recessed portion is flanked by a raised portion at the first side edge, elevated above the recessed portion, and wherein another of the one or more first interlocking features is disposed at the raised portion.

### Example 10

The packaging system of any example herein, in particular example 9, wherein the raised portion is level with the first surface of the first stackable tray.

### Example 11

The packaging system of any example herein, in particular any of examples 8-10, wherein the recessed portion is flanked by a pair of raised portions at the first side edge, with a raised portion at each flank of the recessed portion, each raised portion being elevated above the recessed portion, and wherein a first interlocking feature of the one or more first interlocking features is disposed at each raised portion.

### Example 12

The packaging system of any example herein, in particular example 11, wherein each raised portion is bordered by a subsequent recessed portion that is recessed below the first surface, each subsequent recessed portion having a first interlocking feature of the one or more first interlocking features disposed thereat.

### Example 13

The packaging system of any example herein, in particular any of examples 2-12, wherein the second side edge includes a recessed portion that is recessed below the first surface, and wherein one of the one or more first interlocking features is disposed at the recessed portion.

### Example 14

The packaging system of any example herein, in particular example 13. wherein the recessed portion is flanked by a raised portion at the second side edge, elevated above the recessed portion, and wherein another of the one or more first interlocking features is disposed at the raised portion.

### Example 15

The packaging system of any example herein, in particular any of examples 2-14, wherein the second side edge includes a pair of recessed portions flanking a raised portion at the second side edge, with a recessed portion at each flank of the raised portion, each recessed portion being recessed below the first surface and the raised portion being elevated above the recessed portions, and wherein a first interlocking feature of the one or more first interlocking features is disposed at the raised portion and at each recessed portion.

### Example 16

The packaging system of any example herein, in particular example 15, wherein each recessed portion is bordered by a subsequent raised portion that is elevated above the recessed portions, each subsequent raised portion having a first interlocking feature of the one or more first interlocking features disposed thereat.

### Example 17

The packaging system of any example herein, in particular any of examples 1-16, wherein the one or more first interlocking features each comprises a cavity having an open end, a floor, and a peripheral side wall defining a depth of the cavity.

### Example 18

The packaging system of any example herein, in particular any of examples 1-17, wherein the one or more second interlocking features each comprises a protrusion having a maximal surface and a peripheral side wall defining a height of the protrusion.

### Example 19

The packaging system of any example herein, in particular any of examples 1-18, wherein the first stackable tray is sterile or sterilized.

### Example 20

A packaging system comprising: a first stackable tray for storing one or more implements, the first stackable tray having: a first surface configured to receive the one or more implements; a second surface opposite the first surface; one or more first interlocking features disposed at a first side edge of the first stackable tray at the first surface; and one or more second interlocking features disposed at the first side edge of the first stackable tray at the second surface, the first stackable tray arranged to mate with a second stackable tray via the first interlocking features or the second interlocking features when the first stackable tray is rotated about an axis perpendicular to a plane of the first stackable tray, from a first orientation to a second orientation, relative to the second stackable tray.

### Example 21

The packaging system of any example herein, in particular example 20, further comprising one or more additional first interlocking features disposed at a second side edge of the first stackable tray at the first surface; and one or more additional second interlocking features disposed at the second side edge of the first stackable tray at the second surface.

### Example 22

The packaging system of any example herein, in particular example 21, wherein the second side edge of the first stackable tray includes at least two recessed portions and at least two raised portions, with each recessed portion being recessed below the first surface and each raised portion being elevated above the recessed portions, and wherein an additional first interlocking feature of the one or more additional first interlocking features is disposed at each of the at least two recessed portions and the at least two raised portions.

### Example 23

The packaging system of any example herein, in particular any of examples 21-22, wherein the second stackable tray is identical to the first stackable tray, and wherein one or more first interlocking features of the second stackable tray are arranged to be aligned with and to resiliently engage the one or more second interlocking features of the first stackable tray when the first stackable tray is rotated from the first orientation to the second orientation and the first stackable tray is stacked onto the second stackable tray.

### Example 24

The packaging system of any example herein, in particular any of examples 21-23, wherein the second orientation has a rotational displacement of 180 degrees from the first orientation.

### Example 25

The packaging system of any example herein, in particular any of examples 22-24, wherein the first side edge of the first stackable tray includes at least two recessed portions and at least two raised portions, with each recessed portion being recessed below the first surface and each raised portion being elevated above the recessed portions, and wherein a first interlocking feature of the one or more first interlocking features is disposed at each of the at least two recessed portions and the at least two raised portions.

### Example 26

The packaging system of any example herein, in particular example 25, wherein a pattern of the at least two recessed portions and the at least two raised portions at the first side edge of the first stackable tray comprises an inverse pattern of the at least two recessed portions and the at least two raised portions at the second side edge of the first stackable tray.

### Example 27

The packaging system of any example herein, in particular any of examples 20-26, further comprising a resealable cover, the cover having a plurality of first engagement features on a first surface of the cover arranged to resiliently engage a plurality of second engagement features on the first surface of the first stackable tray to seal the cover to the first surface of the first stackable tray.

### Example 28

The packaging system of any example herein, in particular example 27, wherein the cover includes at least one formed feature arranged to secure the one or more implements within the first stackable tray.

### Example 29

The packaging system of any example herein, in particular any of examples 27-28, wherein a length and a width of the cover fits within a perimeter defined by the one or more first interlocking features at the first surface of the first stackable tray.

### Example 30

The packaging system of any example herein, in particular any of examples 20-29, wherein each first interlocking feature of the one or more first interlocking features is colinear with a second interlocking feature of the one or more second interlocking features on a line parallel to the axis perpendicular to the plane of the first stackable tray.

### Example 31

The packaging system of any example herein, in particular any of examples 20-30, wherein the one or more first interlocking features each comprises a cavity having an open end, a floor, and a peripheral side wall defining a depth of the cavity.

### Example 32

The packaging system of any example herein, in particular any of examples 20-31, wherein the one or more second interlocking features each comprises a protrusion having a maximal surface and a peripheral side wall defining a height of the protrusion.

### Example 33

The packaging system of any example herein, in particular any of examples 20-32, wherein the first stackable tray and the second stackable tray are sterile or sterilized.

### Example 34

The packaging system of any example herein, in particular any of examples 20-33, further comprising a belt configured to secure the first stackable tray to the second stackable tray, the belt including a belt body comprising a flat strap having one or more slots disposed thereon, a latch coupled to or integral with a first end of the belt body via a narrow neck, and a locking tab disposed at a second end of the belt body, the latch having a front end and at least one rear tab and the narrow neck defining a shoulder at the first end of the belt body.

### Example 35

A method, comprising: providing a first stackable tray for storing one or more implements, the first stackable tray having a first surface configured to receive the one or more implements and a second surface opposite the first surface, the first stackable tray including one or more first interlocking features at the first surface of the first stackable tray and one or more second interlocking features at the second surface of the first stackable tray; providing a second stackable tray, the second stackable tray having a first surface including one or more first interlocking features and a second surface opposite the first surface having one or more second interlocking features; rotating the first stackable tray about an axis perpendicular to a plane of the first stackable tray, from a first orientation to a second orientation relative to the second stackable tray; stacking one of the first stackable tray or the second stackable tray on the other of the first stackable tray and the second stackable tray; and aligning and engaging the one or more first interlocking features of the second stackable tray with the one or more second interlocking features of the first stackable tray or the one or more first interlocking features of the first stackable tray with the one or more second interlocking features of the second stackable tray.

### Example 36

The method of any example herein, in particular example 35, wherein each of the first and second stackable trays includes a first side edge and a second side edge, and wherein the one or more first interlocking features are disposed at the first side edge and the second side edge of the first and second stackable trays and the one or more second interlocking features are disposed at the first side edge and the second side edge of the first and second stackable trays.

### Example 37

The method of any example herein, in particular example 36, wherein the aligning comprises mating a recessed portion at the first side edge of one of the first and second stackable trays to a raised portion at the second side edge of the other of the first and second stackable trays.

### Example 38

The method of any example herein, in particular example 36, wherein the first side edge and the second side edge of each of the first and second stackable trays includes at least two recessed portions and at least two raised portions, with each recessed portion being recessed below the first surface of the respective first and second stackable trays and each raised portion being elevated above the recessed portions, and wherein a first interlocking feature of the one or more first interlocking features of the first and second stackable trays is disposed at each of the at least two recessed portions and the at least two raised portions of the first and second stackable trays.

### Example 39

The method of any example herein, in particular example 38, wherein a pattern of the at least two recessed portions and the at least two raised portions at the first side edge of the respective first and second stackable trays comprises an inverse pattern of the at least two recessed portions and the at least two raised portions at the second side edge of the respective first and second stackable trays.

### Example 40

The method of any example herein, in particular any of examples 38-39, wherein a pattern of the at least two recessed portions and the at least two raised portions at the first side edge of the respective first and second stackable trays comprises an alternating pattern of the at least two recessed portions and the at least two raised portions.

### Example 41

The method of any example herein, in particular example 40, wherein a pattern of the at least two recessed portions and the at least two raised portions at the second side edge of the respective first and second stackable trays comprises an inverse pattern of the at least two recessed portions and the at least two raised portions at the first side edge of the respective first and second stackable trays.

### Example 42

The method of any example herein, in particular any of examples 35-41, wherein rotating the first stackable tray from the first orientation to the second orientation comprises rotating the first stackable tray by 90 degrees.

### Example 43

The method of any example herein, in particular any of examples 35-41, wherein rotating the first stackable tray from the first orientation to the second orientation comprises rotating the first stackable tray by 180 degrees.

### Example 44

The method of any example herein, in particular any of examples 35-43, wherein the one or more first interlocking features of the first and second stackable trays each comprises a cavity having an open end, a floor, and a peripheral side wall defining a depth of the cavity.

### Example 45

The method of any example herein, in particular any of examples 35-44, wherein the one or more second interlocking features of the first and second stackable trays each comprises a protrusion having a maximal surface and a peripheral side wall defining a height of the protrusion.

### Example 46

The method of any example herein, in particular any of examples 35-45, wherein the engaging comprises inserting the second interlocking features of the first stackable tray into the first interlocking features of the second stackable tray or inserting the second interlocking features of the second stackable tray into the first interlocking features of the first stackable tray.

### Example 47

The method of any example herein, in particular any of examples 35-46, wherein the second stackable tray is identical to the first stackable tray.

### Example 48

The method of any example herein, in particular any of examples 35-47, further comprising sealing a resealable cover to the first surface of the first stackable tray by resiliently engaging a plurality of first engagement features on a first surface of the cover to a plurality of second engagement features on the first surface of the first stackable tray.

### Example 49

The method of any example herein, in particular any of examples 35-48, wherein each first interlocking feature of the one or more first interlocking features of the first stackable tray is colinear with a second interlocking feature of the one or more second interlocking features of the first stackable tray on a line parallel to the axis perpendicular to the plane of the first stackable tray.

### Example 50

The method of any example herein, in particular any of examples 35-49, wherein the first surface of the first stackable tray includes at least one molded cavity shaped to accommodate the one or more implements.

### Example 51

The method of any example herein, in particular any of examples 35-50, wherein the first stackable tray and the second stackable tray are sterile or sterilized and are configured to retain a sterility of sterile surgical instruments stored therein.

### Example 52

The method of any example herein, in particular any of examples 36-51, wherein the first and second side edges of the respective first and second stackable trays are opposing side edges.

### Example 53

The method of any example herein, in particular any of examples 35-52, further comprising securing the first stackable tray to the second stackable tray using a belt, the belt including a belt body comprising a flat strap having one or more slots disposed thereon and a latch coupled to or integral with a first end of the belt body via a narrow neck, the latch having a front end and at least one rear tab and the narrow neck defining a shoulder at the first end of the belt body; wrapping the belt body around the first and second stackable trays; inserting the latch through one of the one or more slots; engaging the at least one rear tab of the latch to the one of the one or more slots to prevent the latch from backing out of the one of the one or more slots; and engaging a locking tab disposed at a second end of the belt body to the shoulder.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might.” “may,” “e g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having.” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the clement from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A packaging system, comprising:
a first stackable tray for storing one or more implements, the first stackable tray having a first surface configured to receive the one or more implements and a second surface opposite the first surface, the first stackable tray including:
a first side edge including a recessed portion that is recessed below the first surface;
one or more first interlocking features indented into the first surface of the first stackable tray and one or more first interlocking features indented into the recessed portion of the first side edge; and
one or more second interlocking features protruding from the second surface of the first stackable tray, the first stackable tray being arranged to mate with a second stackable tray via engagement of the first interlocking features or the second interlocking features when the first stackable tray is rotated about an axis perpendicular to a plane of the first stackable tray, from a first orientation to a second orientation, relative to the second stackable tray.

2. The packaging system of claim 1, further comprising a second side edge of the first stackable tray including a recessed portion that is recessed below the first surface, wherein one or more first interlocking features is indented into the recessed portion of the the second side edge.

3. The packaging system of claim 2, further comprising one or more first interlocking features indented into the first surface at the second side edge.

4. The packaging system of claim 1, wherein the one or more first interlocking features indented into the first surface include one or more first interlocking features disposed at the first side edge.

5. The packaging system of claim 2, wherein the one or more second interlocking features protrude from the second surface at one or both of the first side edge and the second side edge.

6. The packaging system of claim 5, wherein the one or more second interlocking features protrude from the second surface directly opposite first interlocking features at the first surface.

7. The packaging system of claim 2, wherein the second stackable tray is identical to the first stackable tray, and wherein one or more second interlocking features of the second stackable tray are arranged to be aligned with and to engage the one or more first interlocking features of the first stackable tray when when the first stackable tray is rotated from the first orientation to the second orientation and the second stackable tray is stacked on the first stackable tray.

8. The packaging system of claim 7, wherein rotating the first stackable tray from the first orientation to the second orientation comprises rotating the first stackable tray 90 degrees from the first orientation.

9. The packaging system of claim 7, wherein rotating the first stackable tray from the first orientation to the second orientation comprises rotating the first stackable tray 180 degrees from the first orientation.

10. The packaging system of claim 2, wherein the first and second side edges are opposing side edges.

11. The packaging system of claim 1, wherein the recessed portion at the first side edge is flanked by a raised portion at the first side edge, elevated above the recessed portion, and wherein another of the one or more first interlocking features is indented into the raised portion.

12. A packaging system comprising:
a first stackable tray for storing one or more implements, the first stackable tray having:
a first surface configured to receive the one or more implements;
a second surface opposite the first surface;
one or more first interlocking features indented into a first side edge of the first stackable tray at the first surface; and
one or more second interlocking features protruding from the first side edge of the first stackable tray at the second surface, the first stackable tray arranged to mate with a second stackable tray via the first interlocking features or the second interlocking features when the first stackable tray is rotated about an axis perpendicular to a plane of the first stackable tray, from a first orientation to a second orientation, relative to the second stackable tray.

13. The packaging system of claim 12, wherein the first side edge of the first stackable tray includes at least two recessed portions and at least two raised portions, with each recessed portion being recessed below the first surface and each raised portion being elevated above the recessed portions, and wherein a first interlocking feature of the one or more first interlocking features is indented into each of the at least two recessed portions and the at least two raised portions.

14. The packaging system of claim 13, further comprising a second side edge of the first stackable tray opposite the first side edge of the first stackable tray, wherein a pattern of the at least two recessed portions and the at least two raised portions at the first side edge of the first stackable tray comprises an inverse pattern of at least two recessed portions and at least two raised portions at the second side edge of the first stackable tray.

15. The packaging system of claim 12, wherein the one or more first interlocking features each comprises a cavity having an open end, a floor, and a peripheral side wall defining a depth of the cavity.

16. The packaging system of claim 12, wherein the one or more second interlocking features each comprises a protrusion having a maximal surface and a peripheral side wall defining a height of the protrusion.

17. The packaging system of claim 12, wherein the first stackable tray is sterile or sterilized.

18. A packaging system, comprising:
a first stackable tray for storing one or more implements, the first stackable tray having a first surface configured to receive the one or more implements and a second surface opposite the first surface, the first stackable tray including:
a first side edge including at least one recessed portion and at least one raised portion, with each recessed portion being recessed below the first surface and each raised portion being elevated above the at least one recessed portion;
a first interlocking feature indented into each of the at least one recessed portion and the at least one raised portion; and
one or more second interlocking features protruding from the second surface of the first stackable tray, the first stackable tray being arranged to mate with a second stackable tray via engagement of the first interlocking features or the second interlocking features when the first stackable tray is rotated about an axis perpendicular to a plane of the first stackable tray, from a first orientation to a second orientation, relative to the second stackable tray.

19. The packaging system of any of claim 18, further comprising a resealable cover, the cover having a plurality of first engagement features on a first surface of the cover arranged to resiliently engage a plurality of second engagement features on the first surface of the first stackable tray to seal the cover to the first surface of the first stackable tray.

20. The packaging system of claim 19, wherein the cover includes at least one formed feature arranged to secure the one or more implements within the first stackable tray.

21. The packaging system of claim 18, wherein each first interlocking feature is colinear with a second interlocking feature of the one or more second interlocking features on a line parallel to the axis perpendicular to the plane of the first stackable tray.

22. The packaging system of claim 18, further comprising a belt configured to secure the first stackable tray to the second stackable tray, the belt including a belt body comprising a flat strap having one or more slots disposed thereon, a latch coupled to or integral with a first end of the belt body via a narrow neck, and a locking tab disposed at a second end of the belt body, the latch having a front end and at least one rear tab and the narrow neck defining a shoulder at the first end of the belt body.
